(19) United States
(12) Patent Application Publication
(10) Pub. No.: US 2021/0346188 A1
(43) Pub. Date: Nov. 11, 2021

(54) Wearable Tension Apparatus

(71) Applicant: Alignmed, Inc., Santa Ana, CA (US)

(72) Inventor: William J. Shultz, Newport Beach, CA (US)

(21) Appl. No.: 17/384,688

(22) Filed: Jul. 23, 2021

Related U.S. Application Data
(63) Continuation of application No. 17/228,595, filed on Apr. 12, 2021.
Application No. 17/228,595 is a continuation of application No. 17/177,109, filed on Feb. 16, 2021.
Application No. 17/177,109 is a continuation of application No. PCT/US20/14844, filed on Jan. 23, 2020.
Application No. PCT/US20/14844 is a continuation of application No. PCT/US20/13970, filed on Jan. 17, 2020.
Application No. 17/177,109 is a continuation of application No. PCT/US19/55740, filed on Oct. 10, 2019.
Application No. PCT/US19/55740 is a continuation of application No. PCT/US19/46430, filed on Aug. 13, 2019.
(60) Provisional application No. 62/796,035, filed on Jan. 23, 2019.
(60) Provisional application No. 62/744,002, filed on Oct. 10, 2018.
(60) Provisional application No. 62/718,353, filed on Aug. 13, 2018.
(60) Provisional application No. 62/718,350, filed on Aug. 13, 2018.

(51) Int. Cl. A61F 5/02

(52) U.S. Cl. CPC A61F 5/026

(57) ABSTRACT

Exemplary embodiments contained herein include garments having biomechanical components including neurobands, posture mounts, expansion panels, and combinations thereof.

### BACKGROUND

From birth to death, we are all given one body to live in and help us perform our best. Unfortunately, a wide range of musculoskeletal, neurological and circulatory diseases and disorders exist as a matter of injury, environment, occupation, genetics and disease. A dynamic anatomic garment would provide an interface of therapeutic support and resistance to a body in motion, and thus intervene in the physical maladies associated with injury, arthritis, chronic pain, sensory processing disorder, muscle imbalance, joint misalignment and poor posture.

Women are particularly susceptible given their anatomy and physiology of their upper body. The woman's chest provides a projection of weight that extends in front of the center of gravity and imposes a force distribution unique to women on the upper body. The woman's chest is also made of soft and sensitive tissues that make compression and application of force uncomfortable and sometimes painful. Garments for realigning posture and/or to intervene, influence or otherwise aid the process of body balance, alignment, circulation and/or postural misalignment face hurdles relative to the female upper body anatomy. Conventional garments or taping solutions provide linear applications of pull on the body. But, forces across or around the breast area of a woman can be uncomfortable.

### SUMMARY

Exemplary embodiments of the wearable tension apparatus described herein may include an active mechanism including anatomic arrangements of elastic panels, bands and seams, collectively referred to as neurobands. Neurobands may apply tensile resistance to activate muscles in a kinetic sequence. Thus, resistance on specific muscles may be according to exemplary methods described herein to train, retrain and maintain otherwise activate muscle tissue. This therapeutic approach is unique to use tensile resistance to targeted muscles through a wearable tension garment. An exemplary advantage of such a wearable tension apparatus is to provide a passive therapy which requires no discipline or change in behavior to have measurable effects, outside of periodically wearing a comfortable, form-fit garment—even during sleep. The interface of neurobands with a body in motion provides and extraordinary way to harvest the energy of movement and thus get more out of your body—over suitable periods of time. As such, neurobands may serve as an adjunctive treatment in caregiver strategies for physical rehabilitation, pain management and injury prevention. In occupational environments where repetitive, awkward movements cause injury over time, the garments are designed to be worn comfortably over clothing, as a convenient and economical bio-ergonomic intervention to correspond with the needs of overused and underused muscle groups.

Exemplary embodiments of garments described herein provide for the seamless transfer of forces from a front of the garment to the back of the garment. For example, a garment for use on an upper body may have a seamless transition from the front of the garment to the back of the garment over a shoulder portion of the wearer when in an in use configuration on a wearer.

Exemplary embodiments of garments described herein accommodate the unique physiology of the female anatomy and permit expansion of the garment around and covering the breast area of the wearer. The garment may therefore be configured to create a structural support for the distribution of the frontal weight of the wearer toward the back of the wearer.

Exemplary embodiments described herein include garments worn by woman to influence neuromuscular activity for therapeutic results, or improve physical orientation. An example is a form-fit garment, worn to aid muscle balance, body alignment, circulation and/or postural fitness. A form-fit garment may be used as an adjunctive treatment for chronic pain, injury, disease and disorder. The form-fit garment may also be used to support and bridge to support the frontal weigh imposed by the woman's anatomy toward the back of the wearer. Exemplary embodiments relate generally to a garment or device worn by a person for functional improvements in postural form, health and fitness, comfort, range of motion, reduced interference with range of motion, and combinations thereof.

Exemplary embodiments may include a wearable device, such as a garment, defining a non-linear and/or non-orthogonal construction of anatomic apparel portions used to systematically interact and influence the innate processes of human physiology for improvement in health and fitness.

Exemplary embodiments may comprise dynamic materials with specific elastic resistance, collectively referred to as neurobands. Neurobands may be placed in an interactive matrix to influence the force and direction of body movement and sensory information sent to and from the spinal cord and brain. Thus, a normalization of function can be achieved by reversing or slowing down dysfunctional adaptations that muscles and joints take on with time and injury.

Exemplary methods described herein may use neurobands to apply dynamic tensile resistance and tactile touch to prevent or reduce muscle atrophy by actively enforcing natural movement and neutral anatomy.

Exemplary embodiments described herein may comprise materials having a systematic property of elasticity including single and/or multi-directional orientations. In an exemplary embodiment, materials are knit or woven in an oblique orientation to avoid or reduce perpendicular alignment of the fibers. Exemplary embodiments comprise a warp weave with or without a weft.

In an exemplary embodiment, a garment may include a front panel with greater stretch to permit the expansion or variation of the breast area of of a user. In an exemplary embodiment, the bias of stretch of the front portion of the garment configured to overlay the chest section of the wearer is in a horizontal or across the body direction.

### DRAWINGS

FIG. 1 illustrates a front view of an exemplary embodiment of a garment according to embodiments described herein configured as a half top.

FIG. 2 illustrates a back view of the exemplary embodiment of the garment of FIG. 1.

FIG. 3 illustrates a cutaway view of the garment of FIG. 1 in which the front portion of the garment is removed to show the interior side of the back portion of the garment.

FIG. 4 illustrates an exemplary front view of an exemplary embodiment of a garment according to embodiments described herein configured as a full top.

FIG. 5 illustrates an exemplary front view of an exemplary embodiment of a garment according to embodiments described herein configured as a full top.

### DETAILED DESCRIPTION

In the following description of preferred embodiments, reference is made to the accompanying drawings which form a part hereof, and in which it is shown by way of illustration specific embodiments in which the invention can be practiced. It is to be understood that other embodiments can be used and structural changes can be made without departing from the scope of the embodiments of this invention.

As stated above, the symptoms of a wide variety of human conditions in which muscles, joints, sensory and circulatory systems are compromised, could be helped with an exoskeletal intervention that conforms to the unique environments in which we work and play. The wearable tension apparatus described herein may include a lifestyle technology designed to be comfortable, worn systematically, under or over clothing, over convenient periods of time. Wearable tension-technology is aimed to impact the sociologic and economic burden of musculoskeletal health.

Exemplary embodiments of the wearable tension apparatus described herein includes a garment that fits snugly on the individuals upper body anatomy using conformable elastomeric textiles with specific elastic memory (neurobands) and non-linear construction to address the distinct movements of a human body in both static and dynamic motion. Neurobands provide a multidirectional level of elastic force that is adjoined with a corresponding level of elastic force. Depending on the placement of the neurobands, localized resistance may be applied to the body to train, retrain or maintain afflicted anatomy, thereby resulting in symptom reduction.

Current compression garments do not localize therapeutic resistance to a specific and precise level and consequently do not offer optimal training of over overused and underused muscles. Current compression garments and performance apparel target athletic of physically fit demographics due to the vanity of a form-fit and the difficulty in comfortably fitting anatomy with excessive weight and mass. In addition, the compressing of fat tissue (adipose tissue), does not address a means to reduce dysfunctional adaptions associated with obesity, such as poor postures, early fatigue, muscle imbalance, joint misalignment and compromised circulation. By having high ratios of elastane materials in the pectorals and breast area, and non-linear construction techniques throughout the garment, a conformable expansion of the garment may be achieved at the soft tissue of the breast while maintaining structure integrity to impose support to the body.

Sometimes the neurobands, including panels, bands, and seams vary in location and density to account for the amount of therapeutic force required. The elastomeric properties of each panel, band and seam can be described as having precise anatomic and physiologic purpose.

Exemplary embodiments described herein use neurobands. Exemplary embodiments may include procedures and techniques that use bands of variable stretch characteristics. Exemplary embodiments described herein are in terms of bands, but the invention is not limited to a specific geometric configuration of “band”. Instead, any configuration of a material portion having the desired relative stretch characteristics and/or positions relative to the body are encompassed within the definition of “band”. Accordingly, bands may include linear or elongated pieces, circular pieces, arcs, and any combination of geometric or non-geometric portions as described herein or would be understood by a person of skill in the art.

In an exemplary embodiment, the described procedures and techniques may include positioning and orienting neurobands such that they are anchored to start and end in line with or over corresponding neuromuscular anatomical features. The configuration and position may therefore mirror or correspond to neuromuscular kinesiology or neuromuscular mapping. Exemplary embodiments of neurobands may be used to physically facilitate muscles and their function to support, load, and unload joints. Exemplary embodiments may be used to position joints in certain positions that are in line with good posture and optimal body mechanics. Exemplary embodiments may be used to enhance posture, reduce pain, and aid in recovery.

Exemplary embodiments may be used with garments having a form fit, such as performance apparel, compression apparel, shapewear, maternal wear, among others. Such exoskeletal apparel may produce atypical tensions and pressures which can be restrictive, ill-fitting, ill-positioned, or uncomfortable. Exemplary embodiments may be used to improve anatomic conformability using construction techniques and materials that address the innate properties of human anatomy and physiology. Exemplary embodiments may use, configure, and position materials and component panels to synergistically assist or correspond with contractual properties of the muscle groups in which they overlay, contact, or traverse. Exemplary embodiments may configure material sections to define or correspond to an anatomical form having boarders and/or seams corresponding to anatomical anchoring locations to address the kinetic sequence of the muscle tissue they adjoin. Exemplary embodiments may use, configured, and position materials and component panels to reduce imposed forces or associated discomfort on soft tissue of a wearer. Exemplary embodiments may permit garment expansion to accommodate various anatomies of different wearers without requiring a new garment. A single garment configuration may therefore be used and accommodate many different body shapes.

Exemplary embodiments may incorporate stretch and tensile characteristics that are placed directionally and in line with human kinetics. This may be done using a variety of anchor points on one or more form-fitting posture garments. Neurobands can link the front of the wearer to the back of the wearer to transfer the weight and forces created from the upper body anatomy of a woman to a support infrastructure toward the back of the wearer.

Exemplary embodiments include a garment and method that actively influences the body's own resources to preserve and maintain optimal alignment, that uses biofeedback to enhance the senses of proprioception, kinesthesia, touch and other components of the central, peripheral and somatosensory nerves to maintain and preserve good posture, that uses biofeedback to support optimal body mechanics while simultaneously enhancing muscle memory and muscle retraining, and combinations thereof.

Exemplary embodiments may include neurobands. Exemplary neurobands may include synthetic and/or natural materials with stretch characteristics designed to mimic the contractual properties of muscle tissue for which the neuroband overlays. Exemplary neuroband construction and fiber orientation can be adjusted to assist specific muscles and joints to perform specific functions. Exemplary neurobands may be constructed to allow their forces to be spread over a broad area. Exemplary neurobands may include tensile and tactile properties configured to dynamically provide biofeedback to facilitate muscle and joints movement in the performance of human mobility. Exemplary neurobands may be kinetically placed and may be adjusted to assist muscles and joints in performing specific functions.

Exemplary embodiments may include posture mounts to create attachment points for exemplary neurobands according to embodiments described herein. Posture mounts may be configured to provide anchor points to support the tensile (i.e. pull) function of neurobands according to embodiments described herein. Posture mounts may correspond or overlay tendon and/or ligament locations when the garment is positioned on the wearer. Therefore, posture mounts may provide specific anatomic locations to act as start and stop locations for associated neurobands that overlay corresponding muscle areas of the body. Posture mount attachment may be configured to assist muscle and joints to perform specific functions when one or more neurobands are attached thereto. Posture mounts may define rigid or semi-rigid mounting locations to assist and support attached neurobands.

Exemplary embodiments may include garments constructed to avoid compression in one or more areas of the body. For example, exemplary garments may include expansion panels configured to permit less restrictive movement or reduce pressure on the underlying anatomic features of the body. Exemplary expansion panels may be included over or around the abdomen, breast, or combination thereof. Exemplary expansion panels may include bi-directional or multi-directional stretch orientations to provide greater expansion of the panel over select areas of the body.

Exemplary embodiments of a garment include exemplary seam constructions to coordinate specific elasticity and tensile support throughout the garment. Exemplary seam constructions and placement may correspond to human kinetics and directional transfer of tensile strength of associated neurobands to which the seam may be coupled.

Exemplary embodiments may include a garment according to embodiments described herein. Exemplary garments may include different portions or component parts to define neurobands and/or posture mounts according to embodiments described herein. Exemplary embodiments may include material portions having unidirectional, bidirectional, multi-directional bias of elasticity. Exemplary embodiments may orient the bias of elasticity in specific configuration and orientations to support the underlying body anatomy according to embodiments described herein. Exemplary embodiments may include posture mount locations in which the elasticity of the material is reduced compared to the neurobands such that posture mount locations may position and anchor the neurobands to locations relative to the underlying anatomy of the body.

As used herein, elasticity generally refers to the amount a material can stretch under a given force. Therefore, a greater elasticity indicates a greater amount of stretch when a force is imposed thereon. Rigid or semi-rigid materials are considered to have low elasticity and do not stretch or only include a very limited stretch under an imposition of force. As used herein, the bias of stretch generally indicates that the material has a greater elasticity in the direction of the bias of stretch than in other directions. A material may have one or more than one bias of stretch. Each direction of a material's bias of stretch may have the same elasticity or different elasticities. Therefore, a material may have a major bias of stretch in a first direction and a second bias of stretch in a second direction, where the first bias of stretch has a greater elasticity than the elasticity associated with the second bias of stretch.

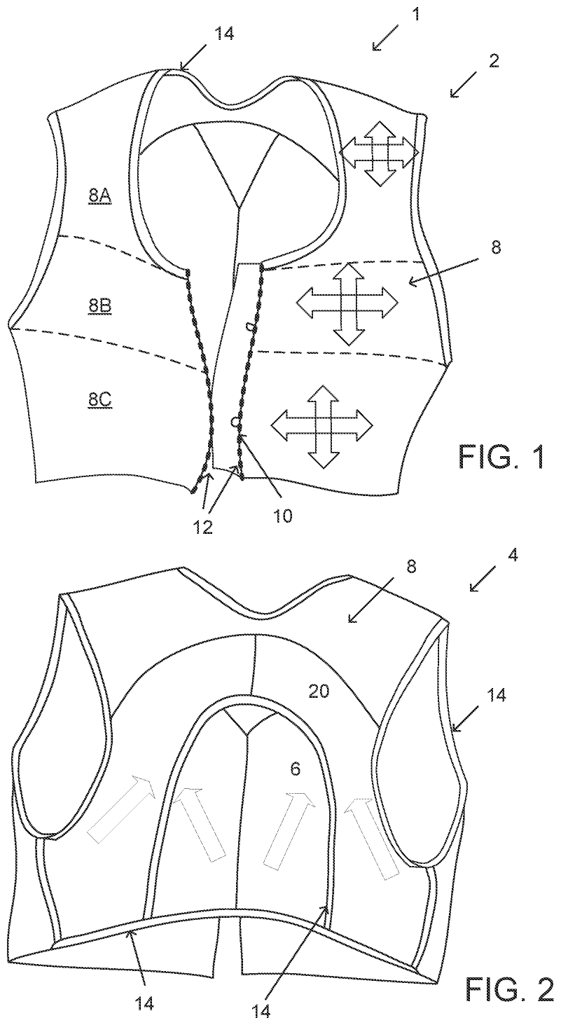

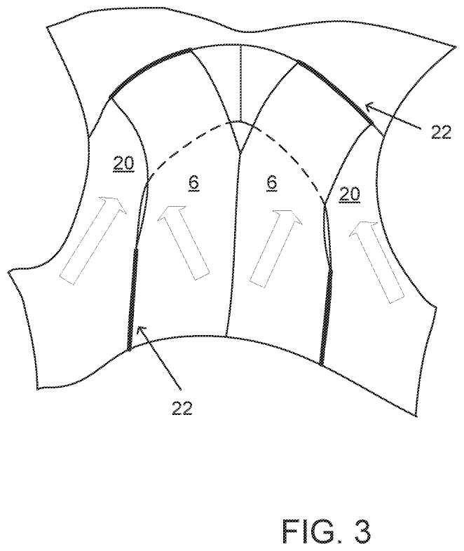

FIGS. 1-3 illustrates an exemplary neuroband configuration for use as a half top. The exemplary garment 1 includes a back portion 4 and a front portion 4 as illustrated in FIGS. 2 and 3, respectively. FIG. 3 illustrates an exemplary view of the interior side of the back side of the garment, corresponding approximately to FIG. 1 with the front portions of the garment including 8A, 8B, 8C removed from view for a better understanding of the interaction of neurobands 6 and 20.

In an exemplary embodiment, the front of the garment 2 may include a single panel that extends from the bottom of the garment, over the chest of a wearer in an in use position, over the shoulder of the wearer or over the top of the garment, traversing from the front to the back of the garment, across the back of the garment, traversing around the back of the neck or upper shoulder/spine of the wearer in an in use configuration, and over the should of the wearer or over the top of the garment, and back down the other front side of the garment, over the chest, and to the bottom of the garment. The front panel 8 may be elastic to accommodate the variability of chest sizes of individual users. In an exemplary embodiment, the front panel 8 may include one or more sections to permit different elasticity within regions of the garment. The sections may be created through various fabric patterns, reduced density fabric areas, reinforcement layers, or in separate panels attached together. In an exemplary embodiment, the front panel 8 is made of a single, unitary panel that incorporates different fabric orientations, densities, patterns, weaves, etc. to create different elasticities in the various sections. For example, the front panel 8 may include two, three, or more sections.

In an exemplary embodiment, a section 8C may correspond to a lower or bottom region of the garment. The first section 8C may have a reduced elasticity compared to one or more other sections to stabilize and create support for the user's soft tissue of the chest and for supporting the rest of the garment including the neurobands described herein. In an exemplary embodiment, the first second 908C may be created by multiple layers of material. For example, two layers may be separately attached and/or a single material panel may be folded over. In an exemplary embodiment, a bottom edge of the garment is created by a fold over of the material that is sewn, bonded, or otherwise attached to the garment to define a lower band.

In an exemplary embodiment, a section 8B may corresponding to an interior portion of the front panel 8 that generally covers the soft tissue of a wearer's breast in an in use position. The second section 8B may have a greater elasticity as compared to one, more, or all of the panels of the garment. The increased elasticity may be created by reducing the material panel layers as compared to, for example, section 8C, or by reducing the fiber density, fiber pattern, or combinations thereof.

In an exemplary embodiment, the front panel 8 may include a section 8A that corresponds to an upper portion of the front panel 8. The top section 8A may be an extension of the section 8B with the same elasticity or may have a different elasticity from the interior portion of section 8B. The top section 8A may extend over the top of the garment and extend from the front of the garment to the back of the garment. The top section 8A may extend around and across the back of the garment. The top section 8A may define and/or be adjacent to the neck aperture and/or at least a portion of the arm aperture of the garment. The top section 8A may have an elasticity between that of the bottom section 8C and the central section 8B. The top section 8A may have an elasticity that is approximately the same as the bottom section 8C or as the central section 8C.

In an exemplary embodiment, the front panel 8 may be separable, and/or attachable. The front panel 8 may include a zipper 12, hook/eye fastener 10, or there combination of connections, such as, for example, buttons, hooks, snaps, hook and loop, etc. In an exemplary embodiment, the garment includes a neck aperture and two arm apertures. In an exemplary embodiment, the apertures include an elastic portion 14 to replace a hem. The hem according to embodiments described herein may include a separate elastic piece folded around the edge of one or more panels to create an edge of the garment. The hem may also include a material bonded or impregnated into the fabric to retain the fabric while permitting elastic flexibility.

FIG. 9B illustrates an exemplary back 4 of the garment according to embodiments described herein including neurobands. As illustrated, the back of the garment includes a plurality of neurobands 6, 20. In an exemplary embodiment, a first pair of neurobands 20 create a boarder or frame for which the second pair of neurobands 6 are positioned. The boarder may be similar to the perimeter 12, neuroband 16, and/or boarder 18 of FIG. 8. The pair of neurobands 20 define an arc, or ovoid opening at the back of the garment. The opening may have a hem 914 as described herein. The pair of neurobands 20 may comprise a directional elasticity. As illustrated by solid arrows, the bias of elasticity of the neuroband 20 is oblique to the vertical axis of the garment that would be positioned along a spine of a wearer in an in use position. The pair of neurobands 20 may be adjacent or define a portion of the arm aperture. The pair of neurobands 20 may couple on the later sides of the garment to the front panel 908 and at the back of the garment along the top of the neurobands 20 to the front panel 8. The top edge traversing the pair of neurobands 20 defines an arc across the back of the wearer in an in use position. The top edge of the neuroband 20 starts in a middle region of the arm aperture and extends upward toward the neck aperture as the top edge is traversed across the garment. In an exemplary embodiment, the top edge approximates the location of the edge of the deltoid, and/or infraspinatus and traverses the trapezius.

In an exemplary embodiment, the garment includes a pair of neurobands 6. The pair of neurobands is configured to be positioned over the trapezius muscle group have a bias of elasticity in an approximately direction of the muscle fibers of the trapezius. As illustrated, the pair of neurobands 6 extend across the aperture created by the pair of neurobands 20 along the bottom of the garment. The pair of neurobands 6 extend upward and are coupled directly together vertically along a vertical center of the garment, configured to be positioned over a spine of a wearer in an in use position. The attachment between neurbands 6 may define an posture mount. The pair of neurbands 6 are unattached along a length of the bands toward the top or toward the neck aperture of the garment. The pair of neurobands are angled away from the vertical axis of the garment corresponding to a spine of a user in an in use configuration and away from each other. The pair of neurobands 6 extend away from each other away from the vertical axis of the garment and up toward the shoulder of the garment where the front panel 8 extends up and over the top of the garment. The pair of neurobands 906 may extend under or over and overlap with the pair of neurobands 2 along a portion of the respective neurobands along a top region of the neurobands. In an exemplary embodiment, the pair of neurobands 6 couple to the front panel 8 at a top portion of the neuroband 906 on the back of the garment 4.

The relationship of an exemplary first pair of neurobands 20 relative to a second pair of neurobands 6 is illustrated by the cut away view of FIG. 3 illustrating an interior side of the back portion of the garment. A first one of a first pair of neurobands 20 extends from a lateral side of the garment and a bottom of the garment upward toward the shoulder of the garment and arcs toward the neck aperture of the garment and toward the central vertical axis of the garment over a spine of the wearer in an in use configuration. A second done of the first pair of neurobands 20 is in mirrored relationship from the first one of the first pair of neurobands 20. The first pair of neurobands creates an arc configuration from the bottom edge of the garment with a separation between the first one and the second one of the first pair of neurobands 20. The second pair of neurobands 6 are positioned within the separation created by the first pair of neurobands 20. A first one of the second pair of neurobands is coupled to the first one of the first pair of neurobands along a length 22 along a lower portion of the respective neurobands. The first one of the second pair of neurobands 6 is coupled along a second length to the second one of the second pair of neurobands 6 along approximately a center, vertical orientation of the garment. The second length of the attachment of the second pair of neurobands 6 to each other may be longer than the attachment length 22 between one of the second pair of neurobands to one of the first pair of neurobands. The second pair of neurobands 6 diverge upwardly and away from each other after the end of the attachment length. The second pair of neurobands 6 are illustrated as extending on an interior side of the first pair of neurobands 20 to create a second layer with the first pair of neurobands. The dotted line indicates the continuation of the first pair of neurobands behind the second pair of neurobands. The second pair of neurobands 6 may traverse a width of the first pair of neurobands to couple to the garment and to the first pair of neurobands at a top or exterior edge of the first pair of neurobands away from the separation space. An exemplary connection location 22 is at a terminal end of the neuroband 6 toward a shoulder of the garment. The first pair of neurobands 20 and second pair of neurobands 6 may be unattached along a length traversing any of the neurobands of the first pair of neurobands. The each of the second pair of neurobands 6 may therefore move locally relative to each of the first pair of neurobands across the width of either of the first pair of neurobands. The first pair of neurobands and second pair of neurobands are statically positioned with respect to one another only along an exterior edge of the neurobands.

Exemplary embodiments of neurobands are described herein. Exemplary embodiments of garment may include any combination of features as described herein. For example, an opening of one embodiment may be combined with one or more neuroband arrangement of another embodiment. The neurobands may be combined in any combination as would be understood by a person of skill in the art and the selected configurations are provided for illustration purposes only.

The garment illustrated in FIGS. 1-3 is configured to support a wearer and transfer the supporting forces for the weight in the front of the body to the posterior side of the body. The garment includes a top section of the garment that extends from a first side of the front of the garment around the back of the garment, across from one side to the other, and then over the front of the garment to the other side of the front of the garment. This section may be used to support a force from the front of the garment to the back of the garment. The back side of the garment is configured with the pair of neurobands to transfer forces to the back of the wearer.

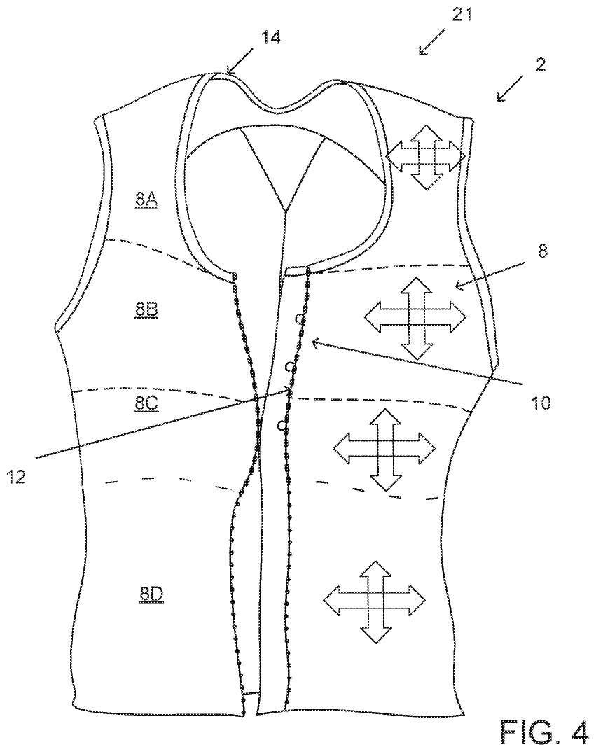

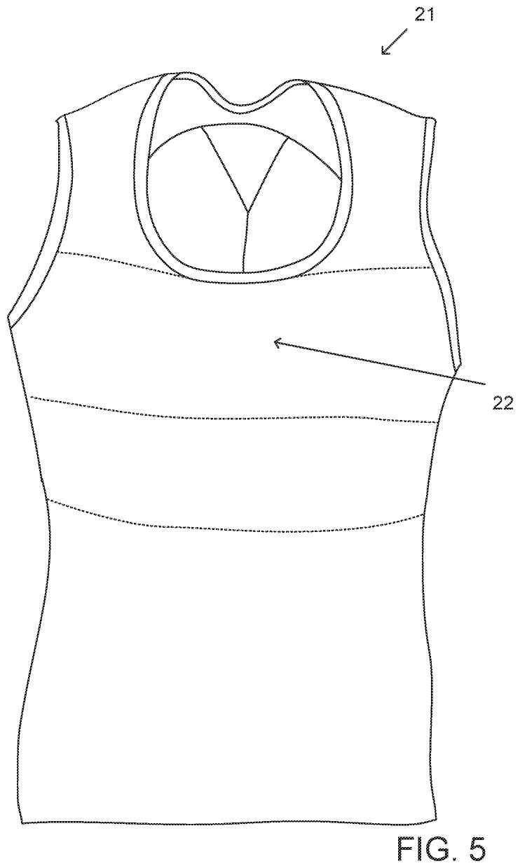

FIG. 4 illustrates an exemplary embodiment in a full shirt configuration. The full shirt configuration may be configured similar to that of the half shirt as illustrated in FIGS. 1-3. The full shirt configuration of a garment 21 may include a front of the garment and a back of the garment. The front of the garment may include a front panel that extends from the bottom of the garment, over the top of the garment, around the back of the garment, and back down on the other side of the front of the garment to the bottom of the garment.

The front panel may include two or more different regions of elasticity. As illustrated, the front of the garment may include four different regions of elasticity 8A, 8B, 8C, 8D traversing from the top of the garment to the bottom of the garment. A given region of elasticity may be different from one or more regions on one or more adjacent sides of a given region. The regions of elasticity may be the same as one or more other regions of elasticity. As illustrated, the regions configured to cover anatomical features of the wearer in an in use position over the abdomen 8D and/or breast 8B of the wearer may include regions of elasticity that are greater than one or more other regions of elasticity. In an exemplary embodiment, the regions at the top of the garment 8A may be less elastic to impose support and pulling forces on the upper body to support the chest of the wearer and transfer the forces to support the wearer from the front of the body to the posterior side of the body or back of the garment. In an exemplary embodiment, the regions under the breast of the wearer in an in use configuration and positioned over a portion of the rib cage of the wearer 8C may have a reduced elasticity than one or more other regions of the front of the garment. The portion over the rib cage may include a region of least elasticity as compared to other regions on the front of the garment. The portion of the garment over the rib cage and under the breast of the wearer may act as an anatomical anchor to support the garment relative to the wearer.

The garment may be configured to separate along a front side of the garment. In an exemplary embodiment, the garment may include a zipper along a front side of the garment. The zipper may be a two way zipper such that the garment may zip and/or unzip from the top and bottom of the garment independent of the other end of the garment. For example, the garment may be fully zipped from the bottom to the top of the garment. A wearer may then be able to unzip the garment a portion of the way from both the bottom and the top ends of the garment, while maintaining the attachment in a middle portion of the garment.

The back of the garment illustrated in FIG. 4 may be similar to the garment illustrated in FIGS. 2-3. The garment may be extended downward, such that the bottom terminal ends of the first and second pair of neurobands simply extend downward to move the terminal end of the garment downward to a lower position on the wearer. Alternatively, the garment may include one or more regions extending from the bottom of the neurobands to define a bottom panel portion of the garment on the back side.

Exemplary embodiments of the garment may include one or more outer layers that may cover any embodiment of the garments described herein. For example, a garment similar to FIGS. 1-3 may include an outer layer positioned over the front, back, or a combination thereof. The outer layer may be coupled to one or more seams or edges of the garment. For example, the outer layer 22 may be positioned over the garment of either FIGS. 1-3 or FIG. 4 and extend from the top of the garment to a bottom of the garment. The top layer 22 may be coupled to the underlying layer(s) at apertures for the neck, and/or aperture for the arms. The top layer 22 may be a form fitting configuration or may be of a larger configuration to permit separation between the outer layer and the body of a wearer in a worn position on the wearer. The top layer 22 may therefore be used to provide a loose cover over the form fitting underlayer(s). The outer layer may also be of a half top length or a full top length. The outer layer may be of the same or different length than the underlying layers. The outer layer may the same length or greater length than underlying layer(s). For illustration purposes only, the underlying layer(s), including different regions of elasticity, are illustrated in dotted lines to indicate presence of the layer under the outer layer 22.

Exemplary embodiments described herein include a component including a neuroband. The component may be integrated or define a garment, a sleeve, or a modular attachment. The garment according to embodiments described herein may also include any combination of a posture mount, an expansion panel, and/or a neuroband, or any combination of one or more of any of these. In an exemplary embodiment, the neuroband comprises at least one bias of elasticity. The garment may include a plurality of neurobands where a first neuroband is configured to wrap around an arm portion of a wearer and define a sleeve, a second neuroband is positioned over a shoulder of a wearer from the front of the garment to a back of the garment. Exemplary embodiments include posture mounts over a spine of a wearer and along the shoulder blade and/or collar bone of a wearer in an in use position.

In an exemplary embodiment, a neuroband is created by overlapping a material to create a multi-layered portion of the garment. The neuroband may include a bias of elasticity and the overlapping material may be oriented such that a first layer and the second layer do not align the bias of elasticity. A fold over of the neuroband may defines a portion of an aperture of the garment, such as, for example, a neck or arm aperture.

Exemplary embodiments of a garment are described herein having a plurality of neurobands, a plurality of posture mounts, a plurality of expansion panels, and combinations thereof. The neurobands may comprise a more elastic material than the posture mount and/or anchor panel. The neurobands may include a bi-directional bias of elasticity. The axis of elasticity of the bi-directional bias of elasticity may be oblique to each other.

In an exemplary embodiment, the garment may include a first pair of neurobands and a second pair of neurobands, wherein the first pair of neurobands overlap the second portion of neurobands. The plurality of neurobands may include a first pair of neurobands and a second pair of neurobands, the first pair of neurobands are configured to define an arcuate shape having a separation between at least a portion of a first one of the first pair of neurobands and at least a portion of a second one of the first pair of neurobands. The second pair of neurobands may be positioned in the separation between the first pair of neurobands. The first pair and second pair of neurobands may include a bias of elasticity. An axis of the bias of elasticity of the first pair and second pair of neurobands may be perpendicular or oblique to each other. The axis of the bias of elasticity between each of the first pair or second pair of neurobands may be perpendicular or oblique to each other. For example, a first one of the first pair of the neurobands may be perpendicular or oblique to either of a second one of the first pair of the neurobands or a first one of the second pair of the neurobands where the first one of the first pair and the first one of the second pair of neurobands are on the same side of the garment and on an opposite side of the garment from the second one of the first pair and the second one of the second pair of neurobands. The a bias of elasticity of the first pair of the neurobands may go toward the center axis of the garment as the garment is traversed either upward or downward, while a bias of elasticity of the second pair of the neurobands may go away from the center axis of the garment as the garment is traversed either upward or downward such that the bias of elasticities of the first pair converge or diverge with respect to each other.

In an exemplary embodiment, a garment having a first pair of neurobands may create an ovoid or arch shape in which a separation is created along a lower edge of the neurobands, and also has a second pair of neurobands that overlap the first pair of neurobands. The first pair of neurobands may be coupled along terminal edges to terminal edges of the second pair of neurobands such that the first pair of neurobands can move relative to the second pair of neurobands along a portion of overlap.

In an exemplary embodiment, the garment may include a front panel that has sections of variable elasticity. The garment may also have a zipper on the front portion of the garment.

Exemplary embodiments include an upper body garment including a front portion and a back portion, the garment comprising a plurality of neurobands. The garment's front portion may be configured to extend from the back portion over the shoulder of a wearer over the breast area of the wearer and to a position under a breast of the wearer at a position over a portion of a rib cage of the wearer in an in use position on a wearer. The garment may be configured as a half top such that it terminates at a lower edge proximate the rib cage of a wearer in the in use position. The front portion may include a panel that extends from the lower edge of the garment on a front first side of the garment, over the breast area of the wearer in the in use position, over the shoulder of the wearer, across a back of the garment and across a neck and spine of the wearer in the in use position, and over a second shoulder of the wearer and down a front second side of the garment over the breast area and to the lower edge of the garment. The panel may be made of a single, unitary material that incorporates different fabric characteristics to create different elasticities in the front portion. The panel may be folded over at the front first side and the front second side at the lower edge of the garment to define the first area of reduced elasticity at the lower edge of the garment. The first area may be at least 2 inches to create a band that provides support for the wearer.

The exemplary garment's front portion may include at least two areas, a first area of reduced elasticity than a second area of greater elasticity, the first area positioned toward the lower edge of the garment than the second area. The garment front portion may include at least three sections of different elasticity compared to an adjacent section.

The exemplary garment's front portion may be separable between the front first side and the front second side of the garment, and the front first side and the front second side are removably attachable.

The exemplary garment may include a first pair of neurobands, wherein each of the first pair of neurobands comprises a directional elasticity. A direction of a major bias of stretch of the directional elasticity is oblique to a vertical axis of the garment in a position on a wearer in an in use position. That garment may also include a second pair of neurobands. The first pair of neurobands are positioned to create an arc defining a space along a bottom edge of the garment, and at least a portion of the second pair of neurobands may be positioned in the space of the arc of the first pair of neurobands. The second pair of neurobands may be directly attached along a length to each other in an area within the arc of the first pair of neurobands. A portion of the first pair of neurobands overlap with a portion of the second pair of neurobands. The second pair of neurobands may be separated from each other in a region that the second pair of neurobands overlap with the first pair of neurobands. The first pair of neurobands may not be attached to the second pair of neurobands for an interior portion of the region where the second pair of neurobands overlap with the first pair of neurobands. The first pair of neurobands may be coupled along terminal edges to terminal edges of the second pair of neurobands such that the first pair of neurobands can move relative to the second pair of neurobands along a region where a portion of one of the first pair of neurobands overlaps a portion of one of the second pair of neurobands. The front portion of the garment may include sections of variable/different elasticity.

Exemplary embodiments described herein may comprise materials having a systematic property of elasticity including single and/or multi-directional orientations. In an exemplary embodiment, materials are knit or woven in an oblique orientation to avoid or reduce perpendicular alignment of the fibers. Exemplary embodiments comprise a warp weave with or without a weft. Exemplary embodiments comprise woven or knit materials infused with an elastic material. Woven or knit materials may include nylon. Elastic materials may include lycra, spandex, elastomer, etc. Exemplary embodiments comprise materials having a warp weave and/or warp knit with and without a weft. In an exemplary embodiment, a weft may comprise titanium strands. An exemplary neuroband may include a dual bias of elasticity. The bi-directional direction of elasticity may permit the neuroband to expand a greater amount in one direction than in another direction under the same application of force. The bi-directional elasticity may permit the neuroband to expand the same amount or different amounts along each of the axis or bias of elasticity. For example, a neuroband may comprise a warp weave having a bi-directional axis of elasticity, where each of the axis of elasticity are oblique to each other. The first axis or bias of elasticity may be greater than the second axis or bias of elasticity such that the material stretches along the first axis by a greater amount than it would along the second axis given the same amount of force. However, the material may stretch less than either of the axis of bias or elasticity in a direction perpendicular to either of the axis of elasticity. In an exemplary embodiment, an exemplary neuroband comprises a bi-directional bias of elasticity. Each of the axis of elasticity are oblique to each other. In an exemplary embodiment, the elasticity along each axis is approximately equal. In an exemplary embodiment, the elasticity along a first axis is approximately 10%-40% more than the elasticity along the second axis.

In an exemplary embodiment, one or more and/or all of the seams of a garment may be bonded. In an exemplary embodiment, one or more and/or all of the seams of the garment may not be sewn. In an exemplary embodiment seams may be bonded by integrating a material into and between the panels of the seam.

Exemplary embodiments of a neuroband, seam, or bond described herein may include woven or knit material infused with an elastic. In an exemplary embodiment, a method of making such an infused material may include providing a fabric material such as a knit or woven material and a sheet of an elastic material. In an exemplary embodiment, the fabric and the sheet may be overlayed over each other. In an exemplary embodiment, the bias of stretch of the sheet and the fabric may be positioned to desired orientation. The bias of stretch of the sheet and the fabric may be parallel, oblique, perpendicular, or other orientation. In an exemplary embodiment, the elastic material is heated to infuse the elastic in the fabric. In an exemplary embodiment, the fabric may comprise nylon or a nylon blend. The elastic may be an elastomer, such as spandex.

Although embodiments of this invention have been fully described with reference to the accompanying drawings, it is to be noted that various changes and modifications will become apparent to those skilled in the art. Such changes and modifications are to be understood as being included within the scope of embodiments of this invention as defined by the appended claims. Specifically, exemplary components are described herein. Any combination of these components may be used in any combination. For example, any component, feature, step or part may be integrated, separated, sub-divided, removed, duplicated, added, or used in any combination and remain within the scope of the present disclosure. Embodiments are exemplary only, and provide an illustrative combination of features, but are not limited thereto.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

1. A upper body garment including a front portion and a back portion, the garment comprising: a plurality of neurobands.

2. The garment of claim 1, wherein the front portion is configured to extend from the back portion over the shoulder of a wearer over the breast area of the wearer and to a position under a breast of the wearer at a position over a portion of a rib cage of the wearer in an in use position on a wearer, the garment is configured to terminate at a lower edge proximate the rib cage of a wearer in the in use position.

3. The garment of claim 2, wherein the front portion of the garment comprises at least two areas, a first area of reduced elasticity than a second area of greater elasticity, the first area positioned toward the lower edge of the garment than the second area.

4. The garment of claim 3, wherein the front portion comprises a panel that extends from the lower edge of the garment on a front first side of the garment, over the breast area of the wearer in the in use position, over the shoulder of the wearer, across a back of the garment and across a neck and spine of the wearer in the in use position, and over a second shoulder of the wearer and down a front second side of the garment over the breast area and to the lower edge of the garment.

5. The garment of claim 4, wherein the panel is made of a single, unitary material that incorporates different fabric characteristics to create different elasticities in the front portion.

6. The garment of claim 5, wherein the garment front portion has at least three sections of different elasticity compared to an adjacent section.

7. The garment of claim 5, wherein the panel is folded over at the front first side and the front second side at the lower edge of the garment to define the first area of reduced elasticity at the lower edge of the garment.

8. The garment of claim 7, wherein the first area is at least 2 inches and creates a band to provide support for the wearer.

9. The garment of claim 1, wherein the front portion is separable between the front first side and the front second side of the garment, and the front first side and the front second side are removably attachable.

10. The garment of claim 1, comprising a first pair of neurobands, wherein each of the first pair of neurobands comprises a directional elasticity.

11. The garment of claim 10, wherein a direction of a major bias of stretch of the directional elasticity is oblique to a vertical axis of the garment in a position on a wearer in an in use position.

12. The garment of claim 11, comprising a second pair of neurobands, and the first pair of neurobands are positioned to create an arc defining a space along a bottom edge of the garment.

13. The garment of claim 12, wherein at least a portion of the second pair of neurobands are positioned in the space of the arc of the first pair of neurobands.

14. The garment of claim 13, wherein the second pair of neurobands are directly attached along a length to each other in an area within the arc of the first pair of neurobands.

15. The garment of claim 14, wherein a portion of the first pair of neurobands overlap with a portion of the second pair of neurobands.

16. The garment of claim 15, wherein the second pair of neurobands are separated from each other in a region that the second pair of neurobands overlap with the first pair of neurobands.

17. The garment of claim 16, wherein the first pair of neurobands are not attached to the second pair of neurobands for an interior portion of the region where the second pair of neurobands overlap with the first pair of neurobands.

18. The garment of claim 16, wherein the first pair of neurobands are coupled along terminal edges to terminal edges of the second pair of neurobands such that the first pair of neurobands can move relative to the second pair of neurobands along a region where a portion of one of the first pair of neurobands overlaps a portion of one of the second pair of neurobands.

19. The garment of claim 1, wherein the front portion of the garment comprises sections of variable elasticity.

20. The garment of claim 19, further comprising a zipper on the front portion of the garment.
